(19) United States
(12) Patent Application Publication
(10) Pub. No.: US 2021/0353509 A1
(43) Pub. Date: Nov. 18, 2021

(54) STOMA POWDER INCLUDING SKIN HEALTH INGREDIENTS

(71) Applicant: Hollister Incorporated, Libertyville, IL (US)

(72) Inventors: George J. Cisko, Spring Grove, IL (US); Jayant Joshi, Lake Forest, IL (US); Joel D. Shutt, Gurnee, IL (US)

(21) Appl. No.: 17/282,276

(22) PCT Filed: Sep. 24, 2019
(86) PCT No.: PCT/US19/52677

§ 371 Date: Apr. 1, 2021

Related U.S. Application Data
(60) Provisional application No. 62/743,938, filed on Oct. 10, 2018.

(51) Int. Cl. A61K 8/02; A61K 31/365; A61K 9/16; A61K 8/73; A61K 8/65; A61K 8/42; A61Q 19/00

(52) U.S. Cl. CPC A61K 8/022; A61K 31/365; A61K 9/1652; A61K 8/73; A61Q 19/005; A61K 9/1617; A61K 8/731; A61K 8/65; A61K 8/42; A61K 9/1658

(57) ABSTRACT

A stoma powder composition is formulated with at least one moisture absorbing material and at least one skin health ingredient. The stoma powder composition is configured to absorb moisture from peristomal skin while improving peristomal skin health.

### BACKGROUND

The present disclosure relates to ostomy accessories, and more particularly to stoma powder.

Stoma powder may be applied to peristomal skin before attaching an ostomy appliance to protect peristomal skin, absorb moisture, and to extend wear time of the ostomy appliance. Many ostomy patients suffer from damaged and irritated skin around a stoma from being in contact with stoma output and using ostomy appliances. Peristomal skin may be compromised by adhesive skin stripping when removing ostomy skin barriers. Active enzymes in stoma discharge may also damage peristomal skin. When damaged or irritated, peristomal skin may weep and become macerated. Stoma powder may be used to absorb excess moisture from weepy and raw skin to promote healing of the skin and to facilitate adhesion of ostomy barriers to skin.

A crusting procedure may be used to absorb moisture from broken skin and provide a dry surface through an artificial scab formed using stoma powder and a liquid film forming composition. The crusting procedure is frequently used on denuded peristomal skin to create a dry surface for attaching an ostomy appliance, while protecting the peristomal skin from stoma output and adhesives. Crusting can increase the time between ostomy appliance changes, resulting in less disruption to irritated peristomal skin.

To form a crusting, a user may clean peristomal skin with water, and pat dry the area. Stoma powder may then be sprinkled on the cleaned peristomal skin. After allowing the dry powder to adhere to the skin, excess powder may be dusted off the skin using a gauze pad or soft tissue. Using a blotting or dabbing motion, a liquid film forming composition, such as Hollister ADAPT® Skin Protective Wipes or Medline MARATHON® Liquid Skin Protectant, may be applied over the powdered peristomal skin area. Alternatively, the liquid skin forming composition may be lightly sprayed over the powdered skin area. The stoma powder and liquid film forming composition application steps may be repeated multiple times to provide a crust over a damaged and/or weeping peristomal skin. An ostomy appliance may be attached to the crust formed peristomal skin.

The present disclosure provides improved stoma powder compositions containing at least one skin health ingredient according to various embodiments.

### BRIEF SUMMARY

A stoma powder composition containing at least one skin health ingredient is provided according to various embodiments. The stoma powder composition may be applied to peristomal skin to absorb moisture and protect skin while improving skin health.

In one aspect, a stoma powder composition may comprise a first powder portion including at least one moisture absorbing material and a second powder portion including at least one skin health ingredient. The stoma powder composition may be configured to absorb moisture from peristomal skin while improving peristomal skin health.

The at least one moisture absorbing material may be selected from the group consisting of hydrocolloids, superabsorbents, and inorganic absorbents. In an embodiment, the first powder portion may comprise pectin, sodium carboxymethyl cellulose, and gelatin. In some embodiments, the first powder portion may have an average particle size of about 15 micron to about 500 micron.

The at least one skin health ingredient may be selected from the group consisting of ceramide, cholesterol, stearic acid, vitamin E, vitamin A, vitamin C, aloe vera extract, fatty acids, anti-inflammatory/soothing agents, such as ginkgolide A, hydrolyzed collagen, antipruritics, antihistamines, alpha-amyrin, beta-amyrin, oleanolic acid, skin hydrating agents like hyaluronic acid, pH buffering agents like citric acid and sodium citrate, super-absorbent powders, antiperspirants, enzyme neutralizing agents, such as zeolites, clays, potato starch derivatives and soybean derivatives. The skin health ingredient may also be selected from one of the microbiome modulating ingredients as a pre-biotic, post-biotic or probiotic agent. In an embodiment, the second powder portion may comprise ceramide.

In an embodiment, the stoma powder composition may comprise about 10 wt. % to about 60 wt. % of pectin, about 10 wt. % to about 60 wt. % of sodium carboxymetylcellulose, about 10 wt. % to about 60 wt. % of gelatin, and about 0.01 w/w% to about 3 w/w% of ceramide.

Other aspects, objectives and advantages will become more apparent from the following detailed description.

### DETAILED DESCRIPTION

While the present disclosure is susceptible of embodiment in various forms, there will hereinafter be described presently preferred embodiments with the understanding that the present disclosure is to be considered an exemplification and is not intended to limit the disclosure to the specific embodiments illustrated.

A stoma power composition including at least one skin friendly ingredient is provided according to various embodiments. The stoma powder composition may be formulated as a non-medicated dry powder composition including at least one skin friendly ingredient and configured to absorb moisture from raw or broken skin surrounding a stoma while improving peristomal skin health, e.g. strengthening, smoothening, soothing, hydrating, and healing. In some embodiments, the stoma powder composition may be configured to form a protective gel when in contact with moisture on skin.

The stoma powder composition may generally comprise at least one moisture absorbing material, and at least one skin health ingredient. Suitable moisture absorbing material may include, but are not limited to, hydrocolloids (e.g. pectin, carboxymethyl cellulose, gelatin, etc.), superabsorbents, inorganic absorbents, and the like.

The at least one skin health ingredient may include materials that protect skin, reduce skin irritation, aid healing, and/or promote skin health. The suitable skin health ingredients may include, but are not limited to, sphingnolipids, including but not limited to ceramides, sphingomyelins, glycosphingolipids, cerebrosides, sulfatides, gangliosides, inositol-containing ceramides and pseudo-ceramides, cholesterol, fatty acids including, but not limited to, palmitic, stearic, oleic, linoleic, and their derivatives such as 12-hydroxystearic acid, 2-ethyl hexyl stearate, etc., vitamins including but not limited to vitamin E (alpha-tocopherol), vitamin C (ascorbic acid), and vitamin D, plant extracts including, but not limited to, extracts of aloe vera (Aloe barbadensis), camu camu (Myrciaria dubia), snow algae (Coenochloris signiensis), Ginko biloba, Artemisia absinthium, Centella asiatica, Siegesbeckia orientalis, Poria cocos, Pseudopterogoria elisabethae, Boswellia serrata, Verbena, officinalis, Glycyrrhiza glabra, willow bark (Salix alba), pomegranate seed, shea butter (Vitellaria pradoxa), Calendula officinalis, anti-inflamatory agents including, but not limited to, hydrocortisone, corticosteroids, non-steroidal anti-inflamatories (NSAIDs), 18B-Glycyrrhetinic Acid (licorice root extract) and its derivatives, stearyl glycrrhetinate, α-bisabolol, limonene, etc., hydrolyzed collagen and collagen protein, antipruritics such as diphenhydramine, hydroxyzine, mint oil, menthol, camphor, lidocaine, benzocaine, benzyl alcohol, etc., alpha-amyrin, beta-amyrin, antiperspirants, such as aluminum chlorohydrate, Alcloxa, etc., pH buffering agents like citric acid and sodium citrate, polyacrylates, enzyme neutralizing agents like potato starch, clays, zeolites or soybean derived ingredients, and microbiome modulating ingredients included as a pre-biotic, post-biotic or a pro-biotic.

Combinations of the suitable skin health ingredients may also be provided. In an embodiment, the at least one skin health ingredient may comprise a blend of ceramide, cholesterol, and stearic acid, such as a ceramide dominant 3:1:1 blend that includes ceramide, cholesterol, and stearic acid in a 3:1:1 ratio.

In an embodiment, the skin health ingredients may be provided in a powder form. In another embodiment, skin health ingredients that are in a liquid form may be incorporated into the stoma powder composition by microencapsulating them in a water-soluble shell made of, for example, gelatin or polyvinyl alcohol. On contact with moisture the shell may dissolve and release the liquid skin care ingredient.

The stoma powder composition may also include other ingredients, such as thickening agents. Suitable thickening agents may include, but are not limited to, gelling agents, such as sodium carboxymetyl cellulose, and others.

In an embodiment, the stoma powder composition may comprise about 10 weight % (wt. %) to about 60 wt. %, preferably about 15 wt. % to about 50 wt. % of pectin, about 10 wt. % to about 60 wt. %, preferably about 15 wt. % to about 50 wt. % of sodium carboxymetylcellulose, about 10 wt. % to about 60 wt. %, preferably about 15 wt. % to about 50 wt. % of gelatin, and about 0.01 wt. % to about 3 wt. %, preferably about .03 wt. % to about 2 wt. % ceramide.

The stoma powder composition may be provided as a powder mixture including a first powder portion comprising at least one moisture absorbing material and a second powder portion comprising at least one skin health ingredient. In an embodiment, the first powder portion may have an average particle size of about 15 microns to about 500 microns, preferably about 20 microns to about 400 microns, and more preferably about 25 microns to about 300 microns.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

1. A stoma powder composition, comprising:
a first powder portion including at least one moisture absorbing material; and
a second powder portion including at least one skin health ingredient;
wherein the stoma powder composition is configured to absorb moisture from peristomal skin while improving peristomal skin health.

2. The stoma powder composition of claim 1, wherein the at least one moisture absorbing material is selected from the group consisting of hydrocolloids, superabsorbents, and inorganic absorbents.

3. The stoma powder composition of claim 1, wherein the first powder portion comprises pectin, sodium carboxymethyl cellulose, and gelatin.

4. The stoma powder composition of claim 1, wherein the at least one skin health ingredient is selected from the group consisting of ceramide, cholesterol, stearic acid, vitamin E, vitamin A, vitamin C, aloe vera extract, and fatty acids, anti-inflammatory/soothing agents, hydrolyzed collagen, antipruritics, antihistamines, alpha-amyrin, beta-amyrin, oleanolic acid, antiperspirants, pH buffering agents, enzyme neutralizing agents, and microbiome modulating ingredients.

5. The stoma powder composition of claim 4, wherein the anti-inflammatory/soothing agents is ginkgolide A.

6. The stoma powder composition of claim 4, wherein the enzyme neutralizing agents are selected from the group consisting of zeolites, clays, potato starch derivatives and soybean derivatives.

7. The stoma powder composition of claim 4, wherein the microbiome modulating ingredients are provided as a pre-biotic agent, a post-biotic agent or a probiotic agent.

8. The stoma powder composition of claim 1, wherein the second powder portion comprises ceramide.

9. The stoma powder composition of claim 1, wherein the stoma powder comprises about 10 wt. % to about 60 wt. % of pectin, about 10 wt. % to about 60 wt. % of sodium carboxymetylcellulose, about 10 wt. % to about 60 wt. % of gelatin, and about 0.01 w/w% to about 3 w/w% of ceramide.

10. The stoma powder composition of claim 1, wherein the first powder portion has an average particle size of about 15 micron to about 500 micron.
